(19) United States
(12) Patent Application Publication
(10) Pub. No.: US 2005/0277624 A1
(43) Pub. Date: Dec. 15, 2005

(54) Use of corticosteroids alone or in conjunction with nutritional therapy to reduce stress in animals

(76) Inventors: Nigel Cook, Innisfail (CA); Allan L. Schaefer, Lacombe (CA); John S. Church, Edmonton (CA)

Correspondence Address: GREENLEE WINNER AND SULLIVAN P C, 4875 PEARL EAST CIRCLE, SUITE 200, BOULDER, CO, 80301, US

(21) Appl. No.: 10/865,671

(22) Filed: Jun. 9, 2004

(51) Int. Cl. A61K 31/573

(52) U.S. Cl. 514/179

(57) ABSTRACT

The invention provides a method of alleviating the effects of stress in a non-human animal comprising administering a therapeutically effective amount of a corticosteroid, such as dexamethasone, alone or in conjunction with a nutritional supplement, to the animal in need thereof. The corticosteroid can be administered prior to exposure to the stress, during the stress, and/or following exposure to the stress. The administration of the corticosteroid in conjunction with a nutritional supplement provides synergistic effects in alleviating the effects of stress, particularly weight loss. The nutrient therapy comprises one or more sources of electrolytes providing each of sodium, potassium and magnesium; one or more sources of amino acids providing each of alanine, lysine, phenylalanine, methionine, threonine, leucine, isoleucine, valine, and glutamate; and a source of tryptophan. The combined administration appears to have a restorative effect.

### FIELD OF THE INVENTION

The invention pertains to the use of corticosteroids alone, or in conjunction with a nutritional supplement to alleviate stress in animals.

### BACKGROUND OF THE INVENTION

Transport and handling to which animals are exposed during standard rearing and marketing practices can be stressful. A plethora of effects has been reported including hypoglycemia, dehydration, energy depletion, protein degradation, electrolyte imbalance and stimulation of the hypothalamic-pituitary-adrenal (HPA) axis (Schaefer et al., 1997; 2001). Such practices may contribute to significant weight loss in addition to other negative effects in animals.

The attenuation of the effects of transport and handling stress has typically involved the examination of the stressors themselves; for example, improvements in animal loading facilities, transport carriers, shock absorbers, wind protection, and loading densities have been recommended, but met with varying levels of success.

An alternative approach involves treatment of the animal rather than the stressor. Attempts have been made to treat the effects of transport stress with a wide variety of different compounds, including amino acids (Japanese Patent Application No. 61-280239 to Takaaki et al.); epinephrine (U.S. Pat. No. 6,133,321 to Prusa et al.); ethyl alcohol (Japanese Patent Application No. 01-113669 to Kosaku et al.); glycerin, licorice roots, vitamins C and E (Russian Patent Nos. 2,160,532, 2,160,533 and 2,153,802 to Ehzergajl et al.); human interleukin-2 (U.S. Pat. No. 4,818,769 to Nunberg, et al.); ionol (Russian Patent No. 2,147,799 to Ajtuev et al.); sedatives such as acepromazine, xylazine and pentobarbitone (Brearly et al., 1990; Sanhouri et al., 1991); and sorbitol (U.S. Pat. No. 5,137,735 to Bignon). In addition, different nutritional therapies have been reported to be effective in reducing transport and handling stress (Schaefer et al., 1997; 2001). U.S. Pat. Nos. 5,505,968 and 5,728,675 to Schaefer et al. issued Apr. 9, 1996 and Mar. 17, 1998 respectively, relates to a nutritional supplement for animals to prevent or reduce antemortem stress.

Reducing weight loss and other deleterious effects in animals during transport or handling stress contributes to both animal welfare and profitability. As existing practices and the prior art do not address the issue of weight loss in animals during transport, there is evidently a need for an effective method or therapy that achieves alleviation of transport stress in animals. Further, commercially available corticosteroids are typically used to treat inflammatory diseases or other illnesses in animals, but current practices for alleviating stress in animals do not involve the use of cortiosteroids alone or in conjunction with a nutritional supplement.

### SUMMARY OF THE INVENTION

The inventors discovered that provision of a corticosteroid to an animal mitigates the effects of stress, particularly weight loss, when a therapeutically effective amount of the corticosteroid is administered to the animal in need thereof. The inventors found that the administration of the corticosteroid in conjunction with a nutritional supplement to the animal provides synergistic effects in alleviating the effects of stress. The combined administration of a corticosteroid and nutritional supplement also was found to have a restorative effect when administered following transport.

Broadly stated, the invention provides a method of alleviating the effects of stress in a non-human animal comprising administering a therapeutically effective amount of a corticosteroid to the animal in need thereof. The invention extends to a method of further administering a corticosteroid in conjunction with a nutrient supplement to the animal. The nutrient supplement comprises:
a) one or more sources of electrolytes providing each of sodium, potassium and magnesium;
b) one or more sources of amino acids providing each of alanine, lysine, phenylalanine, methionine, threonine, leucine, isoleucine, valine, and glutamate; and
c) a source of tryptophan.

As used herein and in the claims, the terms and phrases set out below have the meanings which follow:

“Animal” refers to a non-human animal including, but not limited to, domestic ruminant and monogastric animals, including swine (Sus domesticus), horses, cattle (Bos taurus and Bos indicus); domestic ungulates, including bison, sheep, lamb, deer, moose, elk, caribou and goats; domesticated fowl, including chickens, turkeys, geese, ducks, game birds, and other birds raised in domestication to produce eggs or meat; dogs, cats and other companion animals; wild animals; captured animals; and zoo animals.

“Animal in need thereof” means an animal prior to exposure to the stress, during the stress, and/or following exposure to the stress.

“Antemortem stress” means the stresses imparted to animals during pre-slaughter treatment, including transport, holding, management, and handling.

“Bypass form” means amino acids provided from feed sources such as bypass, chelated or protected proteins. In such forms, the amino acids are not substantially degraded in the rumen of ruminant animals, but pass through to the abomasum comparatively intact. In general, in the context of this invention, an amino acid is considered to be in a bypass form if greater that about 40% of the protein in that feed source is in a bypass form. Without limitation, preferred feed sources of the bypass amino acids include distillers grain, alfalfa meal, corn gluten meal, skim milk powder, whey powder, soybean, caesin, cottonseed meal, feather meal, blood meal, bone and meat meal, and fishmeal.

“Carrier” means a suitable vehicle that is biocompatible and pharmaceutically acceptable, including for instance, one or more solid, semisolid or liquid diluents, excipients, adjuvants, flavours, or encapsulating substances which are suitable for administration.

“Corticosteroids” or “corticosteroid” means the C21 steroid hormones produced by the adrenal cortex. The term also refers to natural analogs and synthetic equivalents of corticosteroids including most preferably, dexamethasone, prednisone, prednisolone, 6α-methylprednisolone, fludrocortisone, triamcinolone, paramethasone, betamethasone, aldosterone, and pharmaceutically acceptable salts thereof.

“Feed” means products including, but not limited to, grasses, legumes, grains, oil seeds, forbes, and sedges, for example oats, barley, wheat, canola, rye, sorghum, millet, corn, molasses, alfalfa, clover, brome, timothy or fescue, bermuda grass, orchard grass, rice straw, and other suitable feedstuffs. The term also includes the above feed sources of the bypass amino acids.

“Hypotonic” means concentration of an ingredient, primarily related to the concentrations of the electrolytes, in an amount that is not significantly greater than the concentration of that ingredient found in the physiological fluids of the animal such as plasma, interstitial and intracellular fluids (i.e. the isotonic concentration). This concentration is preferred so that the supplement provided to the animal will have a lower osmotic pressure in respect of the salts than that of the physiological fluids. Since many animals experiencing stress are dehydrated, the nutrient supplement is preferably formulated to avoid hypertonic liquids (or solids which will result in hypertonic concentrations). Hypertonic solutions would simply draw more fluid from the tissue and exacerbate tissue loss.

“Non-steady state” means a condition in which an animal's endocrine, physiological or metabolic values are in a state of flux often due to environmental factors such as stressors.

“Pharmaceutically- or therapeutically-acceptable” means a substance that does not significantly interfere with the effectiveness of the corticosteroid, or corticosteroid in conjunction with a nutritional supplement and which has an acceptable toxic profile for the animal to which it is administered.

“Pharmaceutically-acceptable salts” means derivatives of the free acid or base forms of the corticosteroids that are modified by addition of appropriate salts.

“Steady-state” means a condition in which an animal's endocrine, physiological and metabolic values are all within a normal range and the animal is not stressed.

“Stress” means environmental stresses imparted to animals during transport stress, antemortem stress, regrouping stress, handling and management stress, mixing of animals, post-parturient stress, weaning stress, acute weather stressors, noise stressors, and when the animal is in a non-steady state. The term also includes other similar animal management stresses.

“Therapeutically effective amount” means any amount of a formulation of the corticosteroid alone, or corticosteroid in conjunction with a nutritional supplement that is sufficient to alleviate effects of stress when administered to the animal in need thereof.

“Transport stress” means stresses imparted to animals during transportation, including for example, handling, weighing, mixing and prods prior to loading; and noise, air temperature, velocity, crowding, pollutants, infectious agents, vibration, motion, and withdrawal of food and water during transport.

### DESCRIPTION OF THE PREFERRED EMBODIMENTS

The invention broadly provides a method of alleviating the effects of stress in a non-human animal comprising administering a therapeutically effective amount of a corticosteroid to the animal in need thereof. Most preferably, dexamethasone is used to alleviate the effects of stress, particularly weight loss. Weight loss generally occurs, for example, during transportation which exposes an animal to a variety of stressors, including handling, weighing, mixing and prods prior to loading; and noise, air temperature, velocity, crowding, pollutants, infectious agents, vibration, motion, and withdrawal of food and water during transport. Dehydration and tissue degradation contributes to significant weight loss in an animal during prolonged transport.

When an animal is exposed to such stressors, it typically responds by displaying activation of the hypothalamic-pituitary-adrenal (HPA) axis, resulting in a substantial increase in the production of an endogenous corticosteroid, namely cortisol (Schaefer and Cook, 2003). Corticosteroids (e.g., cortisol) are steroid hormones produced by the adrenal cortex, and function in metabolic events which are essential for life, including carbohydrate, protein and lipid metabolism; fluid and electrolyte dynamics; energy procurement; and the functions of the cardiovascular system, kidney, skeletal muscle, nervous system and other organs and tissues (Merck, 1973; Haynes, 1990). Cortisol influences numerous biochemical, endocrine and physiological functions to maintain homeostasis of the animal. However, this response subsequently declines under negative feedback control, as cortisol levels are often reported to be relatively low following long-distance transport, indicating fatigue of the HPA axis.

However, the inventors have found that when an animal is supplemented with a corticosteroid having greater potency and biological half-life than cortisol, it is better able to cope physiologically and exhibits less weight loss incurred by stress. Corticosteroid administration in accordance with this invention is most effective for situations in which the animal is exposed to high-energy dependent stressors for prolonged periods (i.e., hours to days). Exposure to cold or hot temperatures and physical activity (i.e., maintaining balance), for example, typically occur during transport. Throughout this period, the ability of the animal's body to supply energy substrates or glucose is reduced due to insufficient levels of cortisol. Without being bound by the same, it is believed that administration of a corticosteroid supplements endogenous corticosteroid levels, and maintains higher levels of energy substrates during periods of peak energy demand.

Dexamethasone is regarded has being approximately 20 times as potent as cortisol in glucocorticoid activity and has a much longer biological half-life (Cook et al., 1993). Dexamethasone is commonly incorporated into pharmaceutical compositions as an anti-inflammatory or anti-infection agent (Haynes, 1990); anti-viral treatment (Chinese Patent No. 1245058 to Zeng et al.); inducer of parturition and lactation (U.S. Pat. No. 3,856,955 to Anderson; U.S. Pat. No. 3,966,927 to Binninger); growth promoter (Meadus et al., 2002, J. Anim. Sci. 74:81-87; United Kingdom Patent No. 2,198,351 to Wilson et al.); alleviator of acute surgical stress (Goodin et al., U.S. Pat. No. 5,670,495); and anti-nausea agent (Tyers et al., U.S. Pat. No. 6,544,550).

However, to the inventors' knowledge, the prior art does not report use of a corticosteroid, such as dexamethasone alone, or in conjunction with a nutritional supplement, to reduce transport stress, particularly the subsequent reduction in weight loss. In the present invention, dexamethasone was proven effective when administered to calves prior to transport to provide energy substrates for a longer period during transport than achieved by the endogenous response. The endogenous response was suppressed by the negative feedback action of dexamethasone on the HPA axis, but the glucocorticoid effects of endogenous cortisol were replaced by the much greater effects of the more powerful, longer acting dexamethasone.

Surprisingly, the inventors found that optimal synergistic effects are obtained when the corticosteroid is used in conjunction with a nutritional supplement. The corticosteroid assists in supplying energy substrates, as mediated by catabolic breakdown of proteins and triglycerides, and Na⁺ ion retention. However, a nutritional supplement contributes essential amino acids and electrolytes necessary to augment the action of the corticosteroid. Further, supplementation with a nutritional supplement appears to facilitate an animal's recovery following transport, in that the animal displays one or more behavioral changes, for example, an improvement in appetite, and a decline in lying frequency.

Corticosteroids, for the purposes of this invention, includes the C21 steroids of the adrenal cortex (for example, cortisol, corticosterone, and aldosterone), natural analogs, and most preferably, synthetic equivalents of corticosteroids including dexamethasone, prednisone, prednisolone, 6α-methylprednisolone, fludrocortisone, triamcinolone, paramethasone, betamethasone, aldosterone, and pharmaceutically-acceptable salts thereof. Corticosteroids are broadly classified as mineralocorticoid or glucocorticoid in accordance with their major function. Mineralocorticoids control salt and water balance through action on the kidneys, whereas glucocorticoids influence synthesis, storage and utilization of glucose. Most corticosteroids display one type of activity or have varying degrees of both types of activity; for example, aldosterone and desoxycorticosterone are highly potent mineralocorticoids, but lack glucocorticoid activity. Cortisol and cortisone are naturally occurring glucocorticoids, yet have weak mineralocorticoid activity. Corticosterone has modest activities in both categories (Haynes, 1990). In the prior art, corticosteroids are primarily used as anti-inflammatory and immunosuppressive agents to treat both humans and animals (Haynes, 1990; Merck, 1973). The relative potency of corticosteroids in terms of their mineralocorticoid, glucocorticoid and anti-inflammatory actions can be expressed relative to cortisol and used to estimate effective minimum doses.

Preferred corticosteroids for use according to the invention include, but are not limited to, dexamethasone, prednisone, prednisolone, 6α-methylprednisolone, fludrocortisone, triamcinolone, paramethasone, betamethasone, aldosterone, and pharmaceutically-acceptable salts thereof. The corticosteroids may be administered according to the invention as solvates thereof or in the form of a pharmaceutically acceptable salt or ester. Suitable salts or esters include the acetate, isonicotinoate, phenylpropionate, pivalate, t-butyl acetate, trioxaundecanoate, disodium metasulphobenzoate and disodium phosphate.

For the present invention, dexamethasone is demonstrated as one example of a synthetic corticosteroid with the ability to alleviate stress in an animal, specifically transport stress. Since corticosteroids essentially share the same functions but to varying degrees, it will be appreciated by those skilled in the art that alternative natural or synthetic corticosteroids similar in structure and function to dexamethasone may be appropriate for the present invention. Further, use of more than one corticosteroid in combination or as a “cocktail” might have appreciable effects by “balancing” the different actions of each corticosteroid.

While the invention is demonstrated as particularly useful for transport stress, it will be appreciated by those skilled in the art that the invention is equally suitable for other stressors involving high-energy demands over prolonged periods and preceded by stressors that initiate HPA axis response. Such types of stress include, but are not limited to, transportation stress, antemortem stress, regrouping stress, handling and management stress, such as moving, herding, walking, branding, neutering, dehorning or antler removal; mixing of animals, post-parturient stress, particularly as applicable to the transition dairy cow; weaning stress; acute weather stressors; noise stressors, particularly acute noise stressors; and on occasions when a non-steady physiological state is being experienced by an animal.

As endogenous corticosteroid response to high-energy demand stressors is similar among all vertebrates, the invention can be applied to a variety of animal species, particularly non-human animals including, but not limited to, domestic ruminant and monogastric animals, including swine (Sus domesticus), horses, cattle (Bos taurus and Bos indicus); domestic ungulates, including bison, sheep, lamb, deer, moose, elk, caribou and goats; domesticated fowl, including chickens, turkeys, geese, ducks, game birds, and other birds raised in domestication to produce eggs or meat; dogs, cats and other companion animals; wild animals; captured animals; and zoo animals.

I. Formulations, Dosages and Administration

a) Corticosteroid Component

Various formulations of the corticosteroid are ideal for administration to animals to mitigate the effects of stress. The corticosteroid can be formulated as a solid, liquid, suspension, aerosol, liquid injectable, topical preparation, feed additive, admixture, and feed composition as follows.

i) Solids

The corticosteroid can be formulated as a solid, capsule, crumble, granule, pellet, pill, powder, tablet and similar dosage form. Corticosteroids may be applied directly into feed bunks or mixed with a ration.

ii) Liquids, Suspensions

The corticosteroid can be incorporated into liquids, formulated as solutions or suspensions by adding powdered corticosteroid to a suitable liquid. A corticosteroid can be mixed with the animal's drinking water or provided in other liquid forms for consumption.

iii) Aerosols

Solutions of the corticosteroid can be converted into aerosols or sprays by standard techniques for making aerosol pharmaceuticals. In general, such techniques comprise pressurizing or providing a means for pressurizing a container of the solution, usually with an inert carrier gas, and passing the pressurized gas through a small orifice. Aerosols can additionally contain customary propellants, for example, inert gases such as nitrogen, carbon dioxide, argon or neon.

iv) Liquid Injectables

The corticosteroid can be incorporated into a sterile injectable solution by adding the corticosteroid in the required amount in an appropriate pharmaceutically- or therapeutically-acceptable carrier (for example, physiological saline or other suitable carriers) followed by filtration, sterilization, or other techniques which ensure that the liquid injectable meets sterility and pyrogenicity requirements for veterinary use. Liquid solutions and formulations of the corticosteroid may lose some activity with aging and are thus either prepared in stable forms, preferably prepared fresh for administration, or include stabilizers, solubilizers and preservatives as are well known, see for example, Goodman and Gilman (1990).

v) Topical Preparations

Topical pharmaceutical compositions, for example creams, lotions, gels, pastes and ointments, and other topical forms such as liposomes, skin patches, etc. can be used for transcutaneous delivery of the corticosteroid to the body.

vi) Feed Additive

The corticosteroid can be administered in the form of a feed additive. The feed additive may be included with the animals' regular feed. Suitable feeds include, but are not limited to, grasses, legumes, grains, oil seeds, forbes, and sedges, for example oats, barley, wheat, canola, rye, sorghum, millet, corn, molasses, alfalfa, clover, brome, timothy or fescue, bermuda grass, orchard grass, rice straw, and other suitable feedstuffs. A feed additive may comprise the corticosteroid in combination with one or more inert or active ingredients.

vii) Admixture

Incorporation of active ingredients into feed material is commonly achieved by preparing a premix of the active ingredient, mixing the premix with vitamins and minerals, and then adding the premix or feed additive to the feed. The corticosteroid can be admixed with other active ingredients known to those in the art. The active ingredients, including the corticosteroid alone or in combination with other active ingredients, can be combined with nutrients to provide a premixed supplement. Nutrients include both micronutrients, such as vitamins, minerals, and macronutrients. The premix may then be added to feed materials.

viii) Feed Composition

The corticosteroid can be provided in the form of a feed composition comprising a feed treated with the corticosteroid. The corticosteroid may be mixed with a feed in dry form; e.g. as a powder, or as a liquid to be used as a drench or spray for example. Suitable feeds include, but are not limited to, grasses, legumes, grains, oil seeds, forbes, and sedges, for example oats, barley, wheat, canola, rye, sorghum, millet, corn, molasses, alfalfa, clover, brome, timothy or fescue, bermuda grass, orchard grass, rice straw, and other suitable feedstuffs.

Typically, the corticosteroid will be formulated in one or more of the forms set out above. These formulations may be stabilized through the addition of proteins or chemical agents. The corticosteroid can be prepared alone or as an active ingredient in pharmaceutical compositions including non-toxic, pharmaceutically or therapeutically acceptable carriers, diluents, excipients, antibiotics, prebiotics, probiotics, micronutrients, vitamins, minerals, and macronutrients, as are well known, see for example, Merck (1973) and Haynes (1990). For standard dosages of conventional pharmacological agents, see for example, Merck (1973). To ensure that the animal consumes a sufficient quantity, flavorings may be added to provide the corticosteroid in a form which appears palatable to the animal. All agents must be non-toxic and pharmaceutically-acceptable for the intended purpose, and must not substantially interfere with the effect of the corticosteroid.

The dosage of the corticosteroid depends upon many factors that are generally known to those skilled in the art, for example, the species, age, and weight of the animal; the choice of corticosteroid and its potency; the type and severity of stress; the time and route of administration; and the type and concentration of the formulation being applied. Appropriate amounts in any given instance will be readily apparent to those skilled in the art or capable of determination by routine experimentation. A therapeutically effective amount of the corticosteroid is desired, namely any amount of a formulation of the corticosteroid which will alleviate the effects of stress when administered to the animal in need thereof. In general, the minimum dose required to mediate a stress modulating effect is the critical factor, and can be estimated from the relative potencies according to Haynes (1990). The maximum dose can be taken as approximately ten times the minimum dose.

The corticosteroid can be administered prior to exposure to the stress, during the stress, and/or following exposure to the stress. Moreover, the corticosteroid can be administered in several ways, including, for example, orally, intramuscularly, intravenously, intra-nasally, via suppository, or transcutaneously, with orally being the most convenient method.

When administered orally (e.g., as a food or water additive), the corticosteroid may be provided, for a 500 kg animal, at a dosage range of 1-20 mg, more preferably 2-5 mg, and most preferably 1-5 mg. For transport stress, oral forms can be administered within 0-48 hours prior to transport, more preferably within 6-12 hours prior to transport, and most preferably within 0-6 hours prior to transport. A longer acting corticosteroid may be administered within 12-24 hours prior to transport.

When administered intramuscularly, the corticosteroid may be provided, for a 500 kg animal, at a dosage range of 1-20 mg, more preferably 2-5 mg, and most preferably 1-5 mg. For transport stress, intramuscular injection of the corticosteroid is conducted within 0 to 48 hours prior to transport, more preferably within 6-12 hours prior to transport, and most preferably 0-6 hours prior to transport. Table 1 provides an example of a dosage schedule for intramuscular administration of common corticosteroids prior to transport. The doses apply to cattle of approximately 1000 lb body weight. The indicated “transport time” refers to the transport period for which the particular corticosteroid is beneficial.

TABLE 1
Dosage Schedule for Intramuscular Administration of Corticosteroids Prior
to Transport or Specific Transport Times

| | Minimum | Transport |
|---|---|---|
| Corticosteroid | Intramuscular Dose (mg) | Time (hr) |
| Dexamethasone | 10 | 0-48 |
| Prednisone | 50 | 0-24 |
| Prednisolone | 50 | 0-24 |
| 6α-methylprednisolone | 50 | 0-24 |
| Fludrocortisone | 20 | 0-12 |
| Triamcinolone | 40 | 0-24 |
| Paramethasone | 20 | 0-48 |
| Betamethasone | 10 | 0-48 |
| Aldosterone | 10 | 0-24 |

When administered intravenously, the corticosteroid may be provided, for a 500 kg animal, at a dosage range of 1-20 mg, more preferably of 2-5 mg, and most preferably of 1-5 mg. For transport stress, intravenous administration of the corticosteroid is conducted within 0 to 48 hours prior to transport, more preferably within 6-12 hours prior to transport, and most preferably 0-6 hours prior to transport. Further, the corticosteroid may be administered intravenously to an animal exhibiting severe signs of distress immediately following transport.

Further, the corticosteroid can be administered intra-nasally using, for example, an aerosol or spray, to deliver the corticosteroid to the animal via the mucosal tissues. However, effective intra-nasal delivery of the corticosteroid is dependent upon several factors, for example, the receptiveness of the animal to aerosol administration with respect to the anatomy and physiology of its nasal cavity, and accessibility of the olfactory region. If intra-nasal administration is unsuitable for small nasal cavities of particular animals, a suppository containing the corticosteroid can be used for placement into an appropriate orifice of the animal where it melts at body temperature, releasing the corticosteroid. A far less invasive technique involves transcutaneous administration. The corticosteroid, in dosage forms suitable for epidermal application (for example, topical preparations such as creams, lotions, gels, pastes, and ointments; liposomes; skin patches; etc.), is thereby applied directly to the skin of the animal for a systemic effect. The dosage ranges and schedules as previously discussed for oral, intramuscular and intravenous administrations are likewise appropriate for intra-nasal, suppository, and transcutaneous administrations.

Approaches for sustaining or controlling release of the corticosteroid include, for example, microspheres, microcapsules, projectile biodegradable missiles and the like. The corticosteroid can be incorporated into a biodegradable polymer which can be injected as a microsphere, for example, reservoir devices (encapsulation of the corticosteroid within a polymer shell) or matrix devices (the corticosteroid is physically entrapped within a polymer network). In addition, implantable controlled-release drug delivery systems, for example, pellets (composed of various polymeric forms), compressed tablets, silastic rubber implants, and silicone capsules, are suitable. The corticosteroid can be incorporated into a biodegradable polymer and the mixture shaped into a disc, fiber or other form for implantation. The implant can then be inserted into the animal through an incision. Examples of implantable devices for sustained or controlled delivery of the corticosteroid within the animal include osmotically or propellant-driven pumps, infusion pumps, mini-pumps, and the like.

b) Corticosteroid in Conjunction with a Nutritional Supplement

Surprisingly, the inventors have found that optimal synergistic effects are obtained when the corticosteroid is used in conjunction with a nutritional supplement. The nutritional supplement for use in the present invention is described in U.S. Pat. Nos. 5,505,968 and 5,728,675 to Schaefer et al. issued Apr. 9, 1996 and March 17 respectively, of which the teachings are applicable to the present invention and are incorporated by reference herein. The nutritional supplement provides the following effects or benefits from the individual ingredients:
i) The electrolyte imbalance from stress is corrected and/or normalized by the inclusion of Na, K and Mg, and preferably bicarbonate.
ii) The hypoglycaemic condition that arises from stress is corrected and/or normalized by the inclusion of a source of energy, preferably glucose, together with the gluconeogenic precursor alanine.
iii) The dehydration associated with stress is corrected and/or normalized either by the inclusion of water in the supplement itself (in the liquid forms of the supplement) or by the provision of water in conjunction with the supplement (in the solid forms of the supplement).
iv) Net protein degradation and carcass loss arising from stress is attenuated by the provision of specific amino acids including leucine, isoleucine and valine, which stimulate protein synthesis and reduce protein degradation, together with the essential amino acids including phenylalanine, lysine, threonine and methionine needed in protein synthesis. For ruminant animals, the amino acids are provided in a bypass form to ensure that they can be utilized by specifically ruminant animals and to ensure that there is a prolonged effect from the supplement after administration to the animal.
v) Hypertension and anxiety experienced from stress is lessened by including the amino acid tryptophan, which is the neurotransmitter precursor to serotonin, together with the blood pressure lowering agent magnesium sulphate. This amino acid is provided in a bypass form for ruminants and/or food grade form.
vi) The effects of meat quality degradation from stress are also lessened by the combined action of electrolytes, which promote acid/base stability and buffering, with NH₃ recipients (glutamate). Protein degradation in animals results in the release of NH₃ groups, which can lead to high pH conditions known to contribute to dark cutting problems in meat quality. The provision of glutamic acid (glutamate) in the supplement alleviates the NH₃ buildup problem. The glutamate, or glutamic acid, is preferably provided in relatively large concentrations, compared to that of other amino acids, to provide an ammonia buffering effect.

Briefly, the nutritional supplement contains an energy source, preferably glucose, up to 1000 g per 500 kg animal. The source of energy is met with the inclusion of simple or complex carbohydrates or fats. Preferable energy sources include one or more of glucose, sucrose, fructose, galactose, dextrose, propylene glycol, lactose, complex carbohydrates such as starch, and fat. Several of these ingredients are beneficially included to delay the effect of the energy source. For instance lactose, starch, propylene glycol, sucrose and fat provide prolonged energy sources. The energy source is preferably provided in a form which is palatable and familiar to the animal. Such sources as kraft whey powder, molasses, and skim milk powder are economic forms of energy sources which are particularly preferred alone, or in admixture with purer energy sources such as glucose, sucrose and dextrose. Other useful energy sources will be evident to persons skilled in the art.

The nutritional supplement also provides Na, K and Mg, preferably at a hypotonic concentration. Ion complexes such as NaCl, KHCO₃ and MgSO₄ are preferred and provide 10 to 20 g of actual ingredient per 500 kg animal.

A source of specific amino acids is provided, including alanine, lysine, phenylalanine, methionine, threonine, leucine, isoleucine, valine and tryptophan at a minimum of 0.5 g per 500 kg animal. Leucine additions at 15 g and glutamate at 40 g per 500 kg animal are preferred. For ruminants, amino acids are provided in a bypass form. The amino acid complex was designed to stimulate protein synthesis, counter protein catabolism and contained the neurotransmitter precursors tryptophan and tyrosine, designed to attenuate the HPA axis response.

The respective amounts of the individual ingredients vary from animal to animal. Generally, as a guide, based on a body weight/three quarters power scale, for a 500 kg ruminant animal consuming about 20 L of water in a day, the preferred ranges of ingredients are as follows (g ingredient/500 kg animal):

| | |
|---|---|
| Energy source (based on glucose) | 20-2000 g (pref. 50-1000) |
| Electrolytes (NaCl, KHCO₃, each) | 2-40 g (pref. 10-20) |
| (MgSO₄) | 1-20 g (pref. 10) |
| Amino Acids (all) | 0.5-100 g (pref. 2-10) |
| (leucine) | 15-25 g |
| (glutamic acid) | 40-66 g |

Exemplary ingredients (if present) are most preferably included in the following percent by weight amounts:

Feed Grade Ingredients
(preferred range is 0.1-4 times the amounts set out below:

| | |
|---|---|
| Flavour | 1% |
| Methionine | 0.5% (as pure source) |
| Lysine | 0.3% (as pure source) |
| Tryptophan | 0.4-1% (as pure source) |
| Threonine | 0.15% (as pure source) |
| Magnesium sulphate | 1-2% (as epsom salts) |
| Potassium chloride | 1.5% |
| Sodium chloride | 2-4% |
| Potassium bicarbonate | 4% |
| Sodium bicarbonate | 4% |
| Dextrose | 20% |
| Animal or Vegetable fat | 2% |
| Molasses | 4% |

Sources of Lactose:

| | |
|---|---|
| Skim milk powder | 2.5-15% |
| Whey powder | 10-20% |

Bypass Proteins as Sources of Amino Acids:

| | |
|---|---|
| Cotton seed meal | 15-50% |
| Corn gluten meal | 15-40% |
| Distillers grain | 30-60% |
| Hydrolysed feather meal | 10-20% |
| Fishmeal | 20-30% |
| Meat and bone meal | 20-40% |
| Blood meal | 10-20% |

Various formulations of the nutritional supplement are fully described in U.S. Pat. Nos. 5,505,968 and 5,728,675 to Schaefer et al., for example, solid feed supplements (preferably as a pelletized solid for admixture with the normal food for the animal), powder premixes for dilution into liquid for either drench or liquid consumable products, or as concentrated liquids for drenches or liquid consumables (with or without dilution). The supplement is most preferably administered as a preventative nutrient supplement before the animal is exposed to the stress. For transport stress, the supplement is preferably administered 6-24 hours prior to transport and most preferably 6-12 hours prior to transport.

Examples 1 and 2 illustrate the effects of corticosteroid alone, and corticosteroid in conjunction with a nutritional supplement upon weight loss normally incurred during transport and handling stress. In Example 1, two treatment groups designated as control, and dexamethasone-treated were studied. In Example 2, three groups designated as control, dexamethasone-treated and a nutritional supplement plus dexamethasone were studied. The results indicate that a corticosteroid, such as dexamethasone, alone and in conjunction with a nutritional supplement is effective in reducing weight loss due to transport and handling stress. Example 3 demonstrates that corticosteroid treatment in conjunction with a nutritional supplement appears to enhance recovery from transport stress, in that animals displayed behavioral changes, namely, an improvement in appetite, and a decline in need to repose. Overall, Examples 1, 2 and 3 suggest that a synthetic corticosteroid, having a potency and biological half-life greater than that of endogenous corticosteroid, can augment the HPA axis response and assist animals in maintaining homeostasis and coping physically with stress.

The invention is illustrated in the following non-limiting examples.

### EXAMPLE 1

### Effects of Corticosteroid Treatment

Forty head of crossbred calves, both steers and heifers, and weighing approximately 600 lb (272 kg) on average were used. The calves were raised at the Agriculture and Agri-Food Canada Lacombe Research Centre (Lacombe, Alberta, Canada) in accordance with standard operating procedures representative of the cattle industry. The calves were randomly divided into two treatment groups designated as control (n=20) and dexamethasone-treated (n=20) and kept on standard feed (cereal grain silage) and water rations prior to transport.

Weights of all animals were recorded pre- and post-transport, metabolic activity was assessed by infrared thermographic analyses (IRT) of the eye region, and a salivary swab collection made for cortisol measurement. The dexamethasone-treated calves were then given an intramuscular dose of dexamethasone at 2 mg per 50 lb (23 kg) body weight.

The animals were subsequently loaded onto a commercial transport and transported for 10 h prior to being off loaded at the Agriculture and Agri-Food Canada Kamloops Research Centre (Kamloops, British Columbia, Canada). On arrival, the calves were again weighed, infrared images captured and a salivary swab collected. The animals were held overnight in pens with ad libitum access to water and cereal grain silage, and were again monitored for weight, thermal changes and salivary cortisol the following morning.

The IRT temperature was significantly lower for the dexamethasone-treated group than the control group following transport (P<0.04). The use of dexamethasone thus results in a lowered thermal response in animals.

For the period of transport between Lacombe and Kamloops (10 h), the control calves lost on average 30.5 lb (13.8 kg or 5% body weight), while the dexamethasone-treated calves lost 19.2 lb (8.7 kg or 3.2% body weight) by comparison. This difference in weight loss was statistically significant (P<0.05). The results suggest that dexamethasone treatment demonstrated the potential to significantly reduce weight loss in transported cattle.

### EXAMPLE 2

### Effects of Corticosteroid Treatment in Conjunction with a Nutritional Supplement

The study in Example 1 was repeated with the addition of a nutritional supplement treatment group. The calves were raised at the Agriculture and Agri-Food Canada Lacombe Research Centre (Lacombe, Alberta, Canada) in accordance with standard operating procedures representative of the cattle industry. The breed, sex and weight of the calves were similar to those used in Example 1. Crossbred calves weighing approximately 800 lb (363 kg) on average were used.

Three treatment groups were designated as control (n=15), dexamethasone-treated or DEX (n=15) and dexamethasone plus nutritional supplement or DEX+NT (n=15). Twenty-four hours prior to transport, the calves were assigned to the nutritional supplement groups and were offered 1 kg/animal of nutritional supplement or a “receiver calf” preparation (Supplement 1(d) as described in U.S. Pat. Nos. 5,505,968 and 5,728,675 to Schaefer et al. with differences being the selection of flavoring, form of an extruded pellet, and carrier being alfalfa) along with their regular feed ration (cereal grain silage). All calves in all treatments were kept on normal feed and water rations prior to transport.

Weights of all animals were recorded pre- and post-transport and metabolic activity was assessed by infrared thermographic analyses of the eye region. The morning of transport all animals were weighed, scanned with infrared cameras and had a salivary swab collected. The dexamethasone treatment given just prior to transport consisted of an intramuscular injection of 0.61 mg per 50 lb body weight. The animals were then loaded onto a commercial transport carrier and transported for 8 h prior to being offloaded, re-weighed, re-scanned with infrared and re-sampled for cortisol. For the DEX+NT animals, the calves were provided on arrival, a further 1 kg/head of a liquid tank mixable and 1 kg/head “receiver calf” pellets in addition to the cereal silage.

The control animals demonstrated a significant increase in temperature over the transport period (P<0.01). Treated animals also exhibited a rise in IRT temperature but to a significantly smaller degree compared to the controls (P<0.05). The use of dexamethasone, or dexamethasone plus nutritional supplement thus results in a lowered thermal response in treated animals.

The control animals lost on average 47.36 lb (21.5 kg or 5.6% body weight) during transport compared to a loss of 38.87 lb (17.6 kg) for the dexamethasone treated calves and a loss of 30.2 lb (13.7 kg or 3.4% body weight) for the combined treatment group of dexamethasone and nutritional supplement. These differences were significantly different at P<0.05. The results indicate that treatment with corticosteroids and with corticosteroids in conjunction with a nutritional supplement resulted in significantly less weight loss in transported cattle. Overall, in both Examples 1 and 2, DEX or DEX+NT groups exhibited significantly less weight losses than Controls (P<0.01). The effects of the nutritional supplement and corticosteroids were synergistic in alleviating transport and handling stress in cattle.

### EXAMPLE 3

### Effects of Corticosteroid Treatment in Conjunction with a Nutritional Supplement on Recovery

Corticosteroid treatment in conjunction with a nutritional supplement appears to enhance recovery from transport stress. The behavioral responses of animals were monitored following transport. Calves were allocated among five treatment groups as follows:

TABLE 2
Treatment Groups to Examine Effects of Corticosteroid Treatment in
Conjunction with a Nutritional Supplement on Recovery

| | # of | |
|---|---|---|
| Group | Animals | Description |
| Control home (CH) | 16 | animals stayed in a pen at the |
| | | Lacombe Research Centre and were |
| | | not exposed to transport |
| Control away (CA) | 15 | control animals exposed to the |
| | | same transport conditions as the |
| | | treatment calves |
| Nutritional supplement | 15 | animals treated with a nutritional |
| (NT) | | supplement prior to transport |
| Nutritional supplement | 15 | animals treated with a nutritional |
| plus dexamethasone | | supplement plus dexamethasone |
| (NT + DEX) | | |
| Dexamethasone alone | 15 | animals treated with dexamethasone |
| (DEX) | | alone |

For all of the above groups, ten behavioural scans lasting one minute each were completed each day (pm) for three days following transport, thereby accumulating a total of 2,280 animal observations. Frequency measures of animals lying and eating were collected. It is well known to those skilled in the art that when animals are fatigued or morbid, they tend to lie down (Kilbour et al., 1984; Agri-Food Research Council Canadian Food Inspection Agency, 2001). Further, animals that are morbid display a reduced feeding frequency, whereas healthy, non-fatigued and hungry animals exhibit an increased feeding frequency.

The CA animals displayed a three day average of 97% lying compared to only 23% in the CH calves. In contrast, the NT calves displayed a 45% lying frequency; the NT+DEX a 39% frequency and the DEX a 0% lying frequency. Such behavioural data suggest that the NT, NT+DEX, and DEX animals all displayed an improved recovery rate based on their reduced need to lie down.

The eating frequency data also supported this conclusion with the three day frequency average for the CA calves at 20% compared to 43%, 35% and 24% in the NT, NT+DEX and DEX animals respectively. In summary, the frequency behaviour results suggest that the DEX, and DEX+NT treated animals recovered faster than controls.

TABLE 3
Results of Corticosteroid Treatment in Conjunction with a Nutritional
Supplement on Recovery

| | Lying | Eating |
|---|---|---|
| Group | Frequency (%) | Frequency (%) |
| Control home (CH) | 23 | 78 |
| Control away (CA) | 97 | 20 |
| Nutritional supplement (NT) | 45 | 43 |
| Nutritional supplement plus | 39 | 35 |
| dexamethasone (NT + DEX) | | |
| Dexamethasone alone (DEX) | 0 | 24 |

### REFERENCES

Agri-Food Research Council. Canadian Food Inspection Agency. Recommended Code of Practice for the Care and Handling of Farm Animals. Transportation 2001. Nepean, Ontario.
Ajtuev, Z. H., Ljapina, V. O., Sen Ko A, J. A., Sizov, F. M., Levakhin, V. I. and Ljapin, O. A. Method of young cattle technological stress prophylaxis. Russian Patent No. 2,147,799.
Anderson, L. L. Method of managing cattle breeding herds. U.S. Pat. No. 4,686,103, issued Aug. 11, 1987.
Bignon, J. Method and feed supplement for reducing the occurrence of dark-cutting meat in animals. U.S. Pat. No. 5,137,735, issued August 11, 1992.
Binninger, C. E. Methods for inducing parturition in cattle with certain intravenously-injected synthetic glucocorticoids U.S. Pat. No. 3,966,927, issued Jun. 29, 1976.
Brearly, J. C., Dobson, H. and Jones, R. S. (1990) Investigations into the effect of two sedatives on the stress response in cattle. J. Vet. Pharmacol. Ther. 13(4):367-77.
Canadian Pharmacists Association. Corticosteroids. In. CPS Compendium of Pharmaceuticals and Specialties, 25^{th} Edition. Canadian Pharmacists Association, Ottawa, pp. 371-379.
Cook, N. J., Read, G. F., Routledge, P. A., Thomas, H., Thevethan, P., Harris, B., Riad-Fahmy, D. (1993) Dexamethasone, pharmokinetics in healthy volunteers assessed by the RIA of dexamethasone in plasma and saliva: Implications for replacement therapy and the dexamethasone suppression tests. In, S. Gorog [Ed.], Advances in Steroid Analysis, 1993, Proceedings of the Fifth Symposium on the Analysis of Steroids,” Szombathely, Hungary.
Ehzergajl, K. V., Gorlov, I. F. and Levakhin, V. I. Cattle transport stress prophylaxis method. Russian Patent No. 2,160,532, published Dec. 20, 2000.
Ehzergajl, K. V., Gorlov, I. F. and Levakhin, V. I. Method for prophylaxis and correcting transport stress of cattle. Russian Patent No. 2,160,533, published Dec. 20, 2000.
Ehzergajl, K. V., Gorlov, I. F. and Levakhin, V. I. Method of prophylaxis and correction of transport stress in cattle. Russian Patent No. 2,153,802, published Aug. 10, 2000.
Goodin, T. H. and Kaufman, R. J. Method of pretreating an animal with a corticosteroid prior to infusion of a perfluorochemical emulsion. U.S. Pat. No. 5,670,495, issued Sep. 23, 1997.
Goodman Gilman, A., Rall, T. W., Niles, A. S. and Taylor, P. (Eds). Goodman and Gilman's The Pharmacological Basic of Therapeutics. 8^{th} Edition. Pergamon Press, N.Y., pp. 1431-1495.
Haynes, R. C., Jr. (1990) Chapter 60: Adrenocorticotropic hormone; adrenocortical steroids and their synthetic analogs; inhibitors of the synthesis and actions of adrenocortical hormones. In Goodman and Gilman's The Pharmacological Basic of Therapeutics. 8^{th} Edition. Eds. A. Goodman Gilman, T. W. Rall, A. S. Niles and P. Taylor. Pergamon Press, N.Y., pp. 1431-1495.
Kilbour, R. and Dalton, D. C. (1984) Livestock Behavior. Metheun Pub. Ltd., Auckland, New Zealand.
Kosaku, F., Akira, T., Morihito, E., Hiroko, H., Taketo, N., Hideo, I. And Yoshimi, U. Transportation of living cattle body. Japanese Patent Application No. 01-113669, published Dec. 6, 1990.
Meadus, W. J., Maclnnis, R., Aalhaus, J. L. and N. J. Cook (2002) Injection of neonatal piglets with dexamethasone stimulates leptin mRNA expression and reduces the stress response in market weight pigs. Anim. Sci. 74:81-87.
The Merck Veterinary Manual (1973) Adrenocortical and related therapy. In A Handbook of Diagnosis and Therapy for the Veterinarian. 4^{th} Edition. Ed. O.H. Siegmund. Merck and Co. Inc., Rathaway, N.J., pp. 587-593.
Nunberg, J. H., Doyle, M., Newell, A. and White, T. Method of controlling stress-related disease in livestock by administration of human IL-2. U.S. Pat. No. 4,818,769, issued Apr. 4, 1989.
Prusa, K. J., Fedler, C. A., Henley, D. and Huskey, L. Method for the reduction of stress in meat producing animals and meat produced from slaughtered animals treated thereby. U.S. Pat. No. 6,133,321, issued Oct. 17, 2000.
Sanhouri, A. A., Jones, R. S. and Dobson, H. (1991) Br. Vet. J. 147(1):42-48.
Schaefer, A. L., Morgan Jones, S. D., Stanley, R. W., Turnbull, I. K. S. and Johanns, J. R.

Antemortem nutrient supplement for animals. U.S. Pat. No. 5,505,968, issued Apr. 9, 1996.
Schaefer, A. L., Morgan Jones, S. D., Stanley, R. W., Turnbull, I. K. S. and Johanns, J. R. Antemortem nutrient supplement for animals. U.S. Pat. No. 5,728,675, issued Mar. 17, 1998.
Schaefer, A. L., Jones, S. D. M. and Stanley, R. W. (1997) The use of electrolyte solutions for reducing transport stress. Anim. Sci. 75:258-265.
Schaefer, A. L., Dubeski, P. L., Aalhus, J. L. and Tong, A. K. W. (2001) Role of nutrition in reducing antemortem stress and meat quality aberrations. J. Anim. Sci. 79(E. Suppl.): E91-E101.
Schaefer, A. L. and Cook, N. J. (2001) The use of amino acids in attenuating HPA response in cervidae. Proc. 7^{th} Int. Con. On Amino Acids and Proteins. August, Univ. of Vienna. Amino Acids 21(1):P32.
Schaefer, A. L. and Cook, N. J. (2003) Stress detection and stress control. Proc. Can. Meat Sci. Assoc. February, Quebec City.
Takaaki, T., Hiroyuki, S., Hirotaka, N. Method of transporting and transferring ruminant. Japanese Patent Application No. 61-280239, published Oct. 12, 1986.
Tyers, M. B. and Challoner, T. E. Medicaments for gastrointestinal disorders. U.S. Pat. No. 6,544,550, issued Apr. 8, 2003.
Wilson, T. J. G., Tivey, D. R., Smith, M. W., James, P. S., Peters, T. J. and Raja, K. B. Improvements in or relating to the use of epidermal growth factor. United Kingdom Patent Application GB 2,198,351, published Jun. 15, 1988.
Zeng, Y. Antivirus veterinary composition. Chinese Patent No. 1245058, published Feb. 23, 2000.

All publications mentioned in this specification are indicative of the level of skill of those skilled in the art to which this invention pertains. All publications are herein incorporated by reference to the same extent as if each individual publication was specifically and individually indicated to be incorporated by reference.

The terms and expressions in this specification are, unless otherwise specifically defined herein, used as terms of description and not of limitation. There is no intention, in using such terms and expressions, of excluding equivalents of the features illustrated and described, it being recognized that the scope of the invention is defined and limited only by the claims which follow.

1. A method of alleviating the effects of stress in a non-human animal comprising: administering a therapeutically-effective amount of a corticosteroid to the animal in need thereof.

2. The method according to claim 1, wherein the stress is selected from the group consisting of transport stress, antemortem stress, regrouping stress, handling and management stress, mixing of animals, post-parturient stress, weaning stress, acute weather stressors, noise stressors, and when the animal is in a non-steady state.

3. The method according to claim 2, wherein the corticosteroid is one or more of dexamethasone, prednisone, prednisolone, 6α-methylprednisolone, fludrocortisone, triamcinolone, paramethasone, betamethasone, aldosterone, and pharmaceutically-acceptable salts thereof.

4. The method as set forth in claim 3, wherein the animal is selected from the group consisting of swine, horses, cattle, bison, sheep, lamb, deer, moose, elk, caribou, goats, chickens, turkeys, geese, ducks, game birds, dogs, cats, companion animals, wild animals, captured animals and zoo animals.

5. The method according to claim 4, wherein the corticosteroid is administered orally, intramuscularly, intravenously, intra-nasally, via suppository, or transcutaneously.

6. The method according to claim 5, wherein the corticosteroid is formulated as a solid, liquid, suspension, aerosol, liquid injectable, topical preparation, feed additive, admixture or feed composition.

7. The method according to claim 6, wherein the corticosteroid is administered at a dosage range of 1 to 20 mg.

8. The method according to claim 6, wherein the corticosteroid is administered at a dosage range of 2 to 5 mg.

9. The method according to claim 6, wherein the corticosteroid is administered at a dosage range of 1 to 5 mg.

10. The method as set forth in claim 6, wherein the animal is cattle.

11. The method according to claim 6, wherein the stress is transport stress.

12. The method according to claim 6, wherein the corticosteroid is dexamethasone.

13. The method according to claim 6, wherein the corticosteroid is in combination with one or more pharmaceutically-acceptable ingredients selected from the group consisting of carriers, diluents, flavorings, excipients, antibiotics, prebiotics, probiotics, micronutrients, vitamins, minerals and macronutrients.

14. The method according to claim 6, wherein the corticosteroid is administered within 0 to 48 hours prior to transport.

15. The method according to claim 6, wherein the corticosteroid is administered within 6 to 12 hours prior to transport.

16. The method according to claim 6, wherein the corticosteroid is administered within 0 to 6 hours prior to transport.

17. The method according to claim 6, further comprising administering the corticosteroid during or following transport.

18. The method according to claim 4, further comprising administering to the animal a nutrient supplement comprising:
a) one or more sources of electrolytes providing each of sodium, potassium and magnesium;
b) one or more feed sources of amino acids providing each of alanine, lysine, phenylalanine, methionine, threonine, leucine, isoleucine, valine, and glutamate; and
c) a source of tryptophan.

19. The method according to claim 18, wherein the amino acids in (b) are in a bypass form, and wherein the animal is a ruminant.

20. The method according to claim 18, wherein each of the sources of amino acids included in the supplement in amounts sufficient to provide, on a dose basis, a total of at least 0.5 g of each amino acid, at least 15 g of leucine, and at least 40 g of glutamate per 1 kg of supplement.

21. The method according to claim 18, wherein each of the sources of amino acids included in the supplement in amounts sufficient to provide, on a dose basis, a total of at least 0.5 g of each amino acid, at least 15 g of leucine, and at least 40 g of glutamate per 500 kg animals per day.

22. The method according to claim 21, wherein the supplement is in a solid or liquid form and is administered to the animals as a feed supplement or drench.

23. The method according to claim 22, wherein the animals being treated are about to be transported or handled and wherein the supplement is administered 6-24 hours prior to transport or handling.

24. The method according to claim 23, wherein the supplement further contains an energy source.

25. The method according to claim 19, wherein the amino acids are provided in bypass form and are provided from one or more of distillers grain, alfalfa meal, corn gluten meal, skim milk powder, whey powder, casein, cottonseed meal, feather meal, blood meal, bone meal, meat meal, and fishmeal.

26. The method according to claim 4, wherein the animal displays one or more of a reduction in weight loss, an improvement in appetite, and a decline in need to repose.
